(19) United States
(12) Patent Application Publication
(10) Pub. No.: US 2008/0241273 A1
(43) Pub. Date: Oct. 2, 2008

(54) MASTIC GUM COMPOSITION FOR USE AS A DIETARY SUPPLEMENT IN HUMANS AND ANIMALS

(75) Inventor: Joseph Scivoletto, Margate, FL (US)

Correspondence Address: DENNIS G. LAPOINTE;LAPOINTE LAW GROUP, PL, PO BOX 1294, TARPON SPRINGS, FL, 34688-1294, US

(73) Assignee: INTACT ENTERPRISES, INC., Margate, FL (US)

(21) Appl. No.: 12/116,319

(22) Filed: May 7, 2008

Related U.S. Application Data
(62) Division of application No. 11/560,374, filed on Nov. 16, 2006.
(60) Provisional application No. 60/754,098, filed on Dec. 27, 2005.

(51) Int. Cl. A61K 36/00; A61K 33/00; A61K 33/24

(52) U.S. Cl. 424/646; 424/725; 424/722

(57) ABSTRACT

A dietary supplement and herbal formula and treatment methodology for humans and animals, having as its primary ingredient mastic gum extract, for the support, increase and production, function, and/or treatment of the kidneys.

### RELATED APPLICATION

This application is a divisional application of U.S. patent application Ser. No. 11/560,374 filed Nov. 16, 2006, which claims the benefit of U.S. provisional patent application Ser. No. 60/754,098 filed Dec. 27, 2005.

### FIELD OF THE INVENTION

The present invention is related generally to a dietary supplement and/or herbal formula and/or herbal agent to be used primarily as a preventative and/or structure and/or function or support and/or treatment of the kidneys, wherein various medical conditions can be improved and treated including hormones of the kidneys, skin, heart, fibrinogen, blood clots, all blood cells including red and white blood cell types and functions, cancer cells, PH balance, acidic blood, anemia, septicemia, hemoglobin, Erythropoietin (EPO, glycoprotein), hematocrit, liver, lactic acid, oxygen to muscles, skeletal muscles, bone, calcitriol (1,25(OH)2 vitamin D3), rickets, sicle cell anemia, immune system enzyme renin, urea protein and cycle, proteins and other macromolecules, colostrum, glucose, omithine transcarbamoylase, Bowman's Capsule, oxygen transport, carbon dioxide transport, ammonia, amino acids, reduction in heart rate, stroke, allergies, Enzymes, AST—Aspartate Transaminase enzymes: AST, Alkaline Phosphotase, CPK, GGT, and the chemicals Bilirubin, blood urea nitrogen (BUN) (a breakdown product of protein metabolism in the blood),Urea and Creatinine.

### BACKGROUND OF THE INVENTION

The following is background information that helps explain terms and problems with the body of humans and animals for which the present invention provides nutritional and remedial benefits for conditions described herein, including blood, organ and neurological disorders.

Blood Substitutes—Years of research have gone into trying to avoid the problems of blood perishability and safety by developing blood substitutes. Most of these have focused on materials that will transport adequate amounts of oxygen to the tissues. Some are totally synthetic substances. Others are derivatives of hemoglobin. Although some have reached clinical testing, none has as yet proved acceptable for routine use.

Because EPO increases the hematocrit, it enables more oxygen to flow to the skeletal muscles. Some cyclists (and distance runners) have used recombinant EPO to enhance their performance. Although recombinant EPO has exactly the same sequence of amino acids as the natural hormone, the sugars attached by the cells used in the pharmaceutical industry differ from those attached by the cells of the human kidney. This difference can be detected by a test of the athlete's urine. Another problem: since recombinant EPO became available, over two dozen young competitive cyclists have died unexpectedly (usually during the night). Perhaps an EPO-induced increase in their hematocrit—leading to a reduction in heart rate—is responsible.

Concerns have been raised in the animal racing (for example, horse racing) as well over the use of recombitant EPO to enhance the performance of the horses. Deaths and anemia have been reported as well in the racing industry. Trainers and others in the racing industry are also concerned about the issues related to the implications of acquiring and administering to horses products known as hormonal growth promotants (HGPs) for typically used for cattle. There have been recent cases where HGPs containing the anabolic steroid trenbolone have been implanted in racehorses. Another area of concern is septicemia a danger to infants , human and animals. It is probably the cause of about 30% of deaths in young foals. Adequate colostrum supplied by the mothers milk, is rich in calories and protein, including antibodies that provide passive immunity for the newborn foal or infant. Colostrum helps starve off septicemia, and other deseases.

What is needed is a safe dietary natural supplement that will provide for support, increase, and production, functional enhancement and/or treatment of various bodily conditions without the need for drugs that have potential adverse effects on the subject, whether it be a human or animal subject.

The invention described below affects the function of the blood. To understand this, the following background is presented relative to blood.

### Blood Performs Two Major Functions:

1. transport through the body of oxygen and carbon dioxide, food molecules (glucose, lipids, amino acids), ions (e.g., Na⁺, Ca²⁺, HCO₃⁻), wastes (e.g., urea), hormones and heat.

2. defense of the body against infections and other foreign materials. All the white blood cells participate in these defenses.

### The Formation of Blood Cells (Cell Types and Acronyms are Defined Below):

All the various types of blood cells are produced in the bone marrow (some 10¹¹ of them each day in an adult human!), and arise from a single type of cell called a hematopoietic stem cell—an “adult” multipotent stem cell. These stem cells are very rare (only about one in 10,000 bone marrow cells); are attached (probably by adherens junctions) to osteoblasts lining the inner surface of bone cavities; express a cell-surface protein designated CD34; and produce, by mitosis, two kinds of progeny: more stem cells (A mouse that has had all its blood stem cells killed by a lethal dose of radiation can be saved by the injection of a single living stem cell!), and cells that begin to differentiate along the paths leading to the various kinds of blood cells.

Which path is taken is regulated by the need for more of that type of blood cell which is, in turn, controlled by appropriate cytokines and/or hormones.

### EXAMPLES

Interleukin-7 (IL-7) is the major cytokine in stimulating bone marrow stem cells to start down the path leading to the various lymphocytes (mostly B cells and T cells).
Erythropoietin (EPO), produced by the kidneys, enhances the production of red blood cells (RBCs).
Thrombopoietin (TPO), assisted by Interleukin-11 (IL-11), stimulates the production of megakaryocytes. Their fragmentation produces platelets.
Granulocyte-macrophage colony-stimulating factor (GM-CSF), as its name suggests, sends cells down the path leading to both those cell types. In due course, one path or the other is taken. Under the influence of granulocyte colony-stimulating factor (G-CSF), they differentiate into neutrophils. Further stimulated by interleukin-5 (IL-5), they develop into eosinophils. Interleukin-3 (IL-3) participates in the differentiation of most of the white blood cells but plays a particularly prominent role in the formation of basophils (responsible for some allergies). Stimulated by macrophage colony-stimulating factor (M-CSF) the granulocyte/macrophage progenitor cells differentiate into monocytes, the precursors of macrophages.

### Red Blood Cells (Erythrocytes)—The Most Numerous Type in the Blood.

Women average about 4.8 million of these cells per cubic millimeter (mm³; which is the same as a microliter (μl)) of blood. Men average about 5.4×10⁶ per μl. These values can vary over quite a range depending on such factors as health and altitude. (Peruvians living at 18,000 feet may have as many as 8.3×10⁶ RBCs per μl.)

RBC precursors mature in the bone marrow closely attached to a macrophage. They manufacture hemoglobin until it accounts for some 90% of the dry weight of the cell. The nucleus is squeezed out of the cell and is ingested by the macrophage. No-longer-needed proteins are expelled from the cell in vesicles called exosomes. Thus RBCs are terminally differentiated; that is, they can never divide. They live about 120 days and then are ingested by phagocytic cells in the liver and spleen. Most of the iron in their hemoglobin is reclaimed for reuse. The remainder of the heme portion of the molecule is degraded into bile pigments and excreted by the liver. Some 3 million RBCs die and are scavenged by the liver each second.

### Red Blood Cells are Responsible for the Transport of Oxygen and Carbon Dioxide.

Oxygen Transport—In adult humans the hemoglobin (Hb) molecule consists of four polypeptides: two alpha (α) chains of 141 amino acids and two beta (β) chains of 146 amino acids. Each of these is attached the prosthetic group heme. There is one atom of iron at the center of each heme. One molecule of oxygen can bind to each heme.

The reaction is reversible under the conditions of lower temperature, higher pH, and increased oxygen pressure in the capillaries of the lungs, the reaction proceeds to the right. The purple-red deoxygenated hemoglobin of the venous blood becomes the bright-red oxyhemoglobin of the arterial blood. Under the conditions of higher temperature, lower pH, and lower oxygen pressure in the tissues, the reverse reaction is promoted and oxyhemoglobin gives up its oxygen.

### Carbon Dioxide Transport

Carbon dioxide (CO₂) combines with water forming carbonic acid, which dissociates into a hydrogen ion (H⁺) and a bicarbonate ions:

CO₂+H₂O [symbol] H₂CO₃ [symbol] H⁺+HCO₃

95% of the CO₂ generated in the tissues is carried in the red blood cells: It probably enters (and leaves) the cell by diffusing through transmembrane channels in the plasma membrane. (One of the proteins that forms the channel is the D antigen that is the most important factor in the Rh system of blood groups.)

Once inside, about one-half of the CO₂ is directly bound to hemoglobin (at a site different from the one that binds oxygen). The rest is converted—following the equation above—by the enzyme carbonic anhydrase into bicarbonate ions that diffuse back out into the plasma and hydrogen ions (H⁺) that bind to the protein portion of the hemoglobin (thus having no effect on pH). Only about 5% of the CO₂ generated in the tissues dissolves directly in the plasma. (A good thing, too: if all the CO₂ we make were carried this way, the pH of the blood would drop from its normal 7.4 to an instantly-fatal 4.5!) When the red cells reach the lungs, these reactions are reversed and CO₂ is released to the air of the alveoli.

### Anemia

Anemia is a shortage of RBCs and/or the amount of hemoglobin in them. Anemia has many causes. One of the most common is an inadequate intake of iron in the diet.

### Blood Groups

Red blood cells have surface antigens that differ between people and that create the so-called blood groups such as the ABO system and the Rh system.

White Blood Cells (leukocytes) are much less numerous than red (the ratio between the two is around 1:700), have nuclei, participate in protecting the body from infection, consist of lymphocytes and monocytes with relatively clear cytoplasm, and three types of granulocytes, whose cytoplasm is filled with granules.

### Lymphocytes

There are several kinds of lymphocytes (although they all look alike under the microscope), each with different functions to perform. The most common types of lymphocytes are B lymphocytes (“B cells”). These are responsible for making antibodies.

T-lymphocytes (“T cells”). There are several subsets of these: inflammatory T cells that recruit macrophages and neutrophils to the site of infection or other tissue damage, cytotoxic T lymphocytes (CTLs) that kill virus-infected and, perhaps, tumor cells helper T cells that enhance the production of antibodies by B cells.

Although bone marrow is the ultimate source of lymphocytes, the lymphocytes that will become T cells migrate from the bone marrow to the thymus where they mature. Both B cells and T cells also take up residence in lymph nodes, the spleen and other tissues where they encounter antigens; continue to divide by mitosis; and mature into fully functional cells.

### Monocytes

Monocytes leave the blood and become macrophages. Macrophages are large, phagocytic cells that engulf foreign material (antigens) that enter the body and dead and dying cells of the body.

### Neutrophils

The most abundant of the WBCs, Neutrophils squeeze through the capillary walls and into infected tissue where they kill the invaders (e.g., bacteria) and then engulf the remnants by phagocytosis. This is a never-ending task, even in healthy people: Our throat, nasal passages, and colon harbor vast numbers of bacteria. Most of these are commensals, and do us no harm. But that is because neutrophils keep them in check.

However, heavy doses of radiation, chemotherapy, and many other forms of stress can reduce the numbers of neutrophils so that formerly harmless bacteria begin to proliferate. The resulting opportunistic infection can be life-threatening.

### Eosinophils

The number of eosinophils in the blood is normally quite low (0-450/μl). However, their numbers increase sharply in certain diseases, especially infections by parasitic worms. Eosinophils are cytotoxic, releasing the contents of their granules on the invader.

### Basophils

The number of basophils also increases during infection. Basophils leave the blood and accumulate at the site of infection or other inflammation. There they discharge the contents of their granules, releasing a variety of mediators such as: histamine, serotonin and prostaglandins and leukotrienes, which increase the blood flow to the area and in other ways add to the inflammatory process. The mediators released by basophils also play an important part in some allergic responses such as hay fever and an anaphylactic response to insect stings.

### Platelets

Platelets are cell fragments produced from megakaryocytes. Blood normally contains 150,000-350,000 per microliter (μl) or cubic millimeter (mm³). This number is normally maintained by a homeostatic (negative-feedback) mechanism. If this value should drop much below 50,000/μl, there is a danger of uncontrolled bleeding because of the essential role that platelets have in blood clotting. Some causes: certain drugs and herbal remedies; and autoimmunity. When blood vessels are cut or damaged, the loss of blood from the system must be stopped before shock and possible death occur. This is accomplished by solidification of the blood, a process called coagulation or clotting. A blood clot consists of a plug of platelets enmeshed in a network of insoluble fibrin molecules.

### Plasma

Plasma is the straw-colored liquid in which the blood cells are suspended.

Composition of blood plasma

| Component | Percent |
|---|---|
| Water | ~92 |
| Proteins | 6-8 |
| Salts | 0.8 |
| Lipids | 0.6 |
| Glucose (blood sugar) | 0.1 |

Plasma transports materials needed by cells and materials that must be removed from cells: various ions (Na⁺, Ca²⁺, HCO₃⁻, etc., glucose and traces of other sugars, amino acids, other organic acids, cholesterol and other lipids, hormones and urea and other wastes. Most of these materials are in transit from a place where they are added to the blood (a “source”) exchange organs like the intestine and depots of materials like the liver to places (“sinks”) where they will be removed from the blood.

### Serum Proteins

Proteins make up 6-8% of the blood. They are about equally divided between serum albumin and a great variety of serum globulins. After blood is withdrawn from a vein and allowed to clot, the clot slowly shrinks. As it does so, a clear fluid called serum is squeezed out. Thus: Serum is blood plasma without fibrinogen and other clotting factors. The serum proteins can be separated by electrophoresis. A drop of serum is applied in a band to a thin sheet of supporting material, like paper, that has been soaked in a slightly-alkaline salt solution. At pH 8.6, which is commonly used, all the proteins are negatively charged, but some more strongly than others. A direct current can flow through the paper because of the conductivity of the buffer with which it is moistened. As the current flows, the serum proteins move toward the positive electrode. The stronger the negative charge on a protein, the faster it migrates. After a time (typically 20 min), the current is turned off and the proteins stained to make them visible (most are otherwise colorless). The separated proteins appear as distinct bands.

The most prominent of these and the one that moves closest to the positive electrode is serum albumin. Serum albumin is made in the liver binds many small molecules for transport through the blood, helps maintain the osmotic pressure of the blood.

The other proteins are the various serum globulins. They migrate in the order alpha globulins (e.g., the proteins that transport thyroxine and retinol [vitamin A]), beta globulins (e.g., the iron-transporting protein transferrin) and gamma globulins. Gamma globulins are the least negatively-charged serum proteins. (They are so weakly charged, in fact, that some are swept in the flow of buffer back toward the negative electrode.) Most antibodies are gamma globulins. Therefore gamma globulins become more abundant following infections or immunizations.

If an antibody-secreting cell—called a plasma cell—becomes cancerous, it grows into a clone secreting a single kind of antibody molecule. Gamma globulins can be harvested from donated blood (usually pooled from several thousand donors) and injected into persons exposed to certain diseases such as chicken pox and hepatitis. Because such preparations of immune globulin contain antibodies against most common infectious diseases, the patient gains temporary protection against the disease.

### Serum Lipids

Because of their relationship to cardiovascular disease, the analysis of serum lipids has become an important health measure. The table shows the range of typical values as well as the values above (or below) which the subject may be at increased risk of developing atherosclerosis.

| LIPID | Typical values (mg/dl) | Desirable (mg/dl) |
|---|---|---|
| Cholesterol (total) | 170-210 | <200 |
| LDL cholesterol | 60-140 | <100 |
| HDL cholesterol | 35-85 | >40 |
| Triglycerides | 40-160 | <160 |

Total cholesterol is the sum of HDL cholesterol, LDL cholesterol and 20% of the triglyceride value. Note that high LDL values are bad, but high HDL values are good. Using the various values, one can calculate a cardiac risk ratio=total cholesterol divided by HDL cholesterol. A cardiac risk ratio greater than 7 is considered a warning.

### Blood Transfusions

In the United States, in 2001, some 15 million “units” (475 ml) of blood were collected from blood donors. Some of these units (“whole blood”) were transfused directly into patients (e.g., to replace blood lost by trauma or during surgery). Most were further fractionated into components, including: RBCs. When refrigerated these can be used for up to 42 days.

Platelets: These must be stored at room temperature and thus can be saved for only 5 days. Plasma: This can be frozen and stored for up to a year.

### Ensuring the Safety of Donated Blood.

A variety of infectious agents can be present in blood. Viruses (e.g., HIV-1, hepatitis B and C, HTLV, West Nile virus bacteria like the spirochete of syphilis, protozoans like the agents of malaria and babesiosis. Prions (e.g., the agent of variant Crueutzfeldt-Jakob disease) and could be transmitted to recipients. To minimize these risks, donors are questioned about their possible exposure to these agents; each unit of blood is tested for a variety of infectious agents. Most of these tests are performed with enzyme immunoassays (EIA) and detect antibodies against the agents. However, it takes a period of time for the immune system to produce antibodies following infection, and during this period (“window”), infectious virus is present in the blood. For this reason, blood is now also checked for the presence of the RNA of these RNA viruses: HIV-1, hepatitis C, West Nile virus, by the so-called nucleic acid-amplification test (NAT).

Thanks to all these precautions, the risk of acquiring an infection from any of these agents is vanishingly small. Despite this, some people—in anticipation of need—donate their own blood (“autologous blood donation”) prior to surgery.

### Blood Typing

Donated blood must also be tested for certain cell-surface antigens that might cause a dangerous transfusion reaction in an improperly-matched recipient.

### Blood Substitutes

Years of research have gone into trying to avoid the problems of blood perishability and safety by developing blood substitutes. Most of these have focused on materials that will transport adequate amounts of oxygen to the tissues. Some are totally synthetic substances. Others are derivatives of hemoglobin. Although some have reached clinical testing, none has as yet proved acceptable for routine use.

pH is a measure of the concentration of hydrogens ions (═H⁺) (=protons) in a solution. Numerically it is the negative logarithm of that concentration expressed in moles per liter (M). Pure water spontaneously dissociates into ions, forming a 10⁻⁷ M solution of H⁺ (and OH⁻). The negative of this logarithm is 7, so the pH of pure water is 7. Solutions with a higher concentration of H⁺ than occurs in pure water have pH values below 7 and are acidic. Solutions containing molecules or ions that reduce the concentration of H⁺ below that of pure water have pH values above 7 and are basic or alkaline.

Is pH important? Yes! The properties of most proteins, enzymes for example, are sensitive to pH. As the pH drops, H⁺ bind to the carboxyl groups (COO⁻) of aspartic acid (Asp) and glutamic acid (Glu), neutralizing their negative charge, and H⁺ bind to the unoccupied pair of electrons on the N atom of the amino (NH₂ ) groups of lysine (Lys) and arginine (Arg) giving them a positive charge.

The result: Not only does the net charge on the molecule change (it becomes more positive) but many of the opportunities that its R groups have for ionic (electrostatic) interactions with other molecules and ions are altered. As the pH rises, H⁺ are removed from the COOH groups of Asp and Glu, giving them a negative charge (COO⁻), and H⁺ are removed from the NH₃⁺ groups of Lys and Arg removing their positive charge.

The result: Again the net charge on the molecule changes (it becomes more negative) and, again, many of the opportunities its R groups have for electrostatic interactions with other molecules or ions are altered.

The pH of the cytosol within a human cell is about 7.4. BUT, this value masks the pH differences that are found in various compartments within the cell. For example, the interior of lysosomes is much more acidic (as low as pH 4) than the cytosol, and the enzymes within work best at these low pH values. The pH differential created within chloroplasts by the energy of the sun is harnessed to synthesize ATP which, in turn, powers the synthesis of food. The pH differential created within mitochondria during the respiration of food is harnessed to the synthesis of ATP which, in turn, powers most of the energy-consuming activities of the cell such as locomotion and biosynthesis of cell components.

### Hormones of the Kidney, Skin, and Heart

### Kidney.

The human kidney secretes two hormones: Erythropoietin (EPO) and Calcitriol (1,25[OH]₂ Vitamin D₃) as well as the enzyme renin. Erythropoietin (EPO) is a glycoprotein. It acts on the bone marrow to increase the production of red blood cells. Stimuli such as bleeding or moving to high altitudes (where oxygen is scarcer) trigger the release of EPO. People with failing kidneys can be kept alive by dialysis. But dialysis only cleanses the blood of wastes. Without a source of EPO, these patients suffer from anemia. Now, thanks to recombinant DNA technology, recombinant human EPO is available to treat these patients.

Some other uses for recombinant EPO: Some of the drugs used to treat AIDS, zidovudine (AZT) for example, cause anemia as a side effect. Recombinant EPO helps AIDS patients cope with this one of the many problems that the disease creates. Recombinant EPO improves the anemia that is such a frequent side effect of cancer chemotherapy. Severe blood loss in Jehovah's Witnesses, whose religion forbids them to receive blood transfusions, can also be helped with recombinant EPO.

Because EPO increases the hematocrit, it enables more oxygen to flow to the skeletal muscles. Some cyclists (and distance runners) have used recombinant EPO to enhance their performance. Although recombinant EPO has exactly the same sequence of amino acids as the natural hormone, the sugars attached by the cells used in the pharmaceutical industry differ from those attached by the cells of the human kidney. This difference can be detected by a test of the athlete's urine.

Another problem: since recombinant EPO became available, over two dozen young competitive cyclists have died unexpectedly (usually during the night). Perhaps an EPO-induced increase in their hematocrit—leading to a reduction in heart rate—is responsible. Recently it has been found that EPO is also synthesized in the brain when oxygen becomes scarce there (e.g., following a stroke), and helps protect neurons from damage. Perhaps recombinant human EPO will turn out to be useful for stroke victims as well.

### Calcitriol

Calcitriol is 1,25[OH]₂ Vitamin D₃, the active form of vitamin D. It is derived from calciferol (vitamin D₃) which is synthesized in skin exposed to the ultraviolet rays of the sun, precursors (“vitamin D”) ingested in the diet. Calciferol in the blood is converted into the active vitamin in two steps: calciferol is converted in the liver into 25[OH] vitamin D₃ this is carried to the kidneys (bound to a serum globulin) where it is converted into calcitriol. This final step is promoted by the parathyroid hormone (PTH).

### Calcitriol Action

Calcitriol acts on the cells of the intestine to promote the absorption of calcium from food and bone to mobilize calcium from the bone to the blood. Calcitriol enters cells and, if they contain receptors for it (intestine cells do), it binds to them. The calcitriol receptors are zinc-finger transcription factors. The receptor-ligand complex bind to its response element, the DNA sequence: 5′ AGGTCAnnnAGGTCA 3′. This sequence of nucleotides (n can be any nucleotide) is found in the promoters of genes that are turned on by calcitriol. Once the hormone-receptor complex is bound to its response element, other transcription factors are recruited to the promoter and transcription of the gene(s) begins.

### Deficiency Disorders

Insufficient calcitriol prevents normal deposition of calcium in bone. In childhood, this produces the deformed bones characteristic of rickets. In adults, it produces weakened bones causing osteomalacia. The most common causes are inadequate amounts of the vitamin in the diet or insufficient exposure to the sun. However, some rare inherited cases turn out to be caused by inheriting two mutant genes for the kidney enzyme that converts 25[OH] vitamin D₃ into calcitriol. Other cases of inherited rickets (also very rare) are caused by inheriting two defective genes for the calcitriol receptor. Mutations that change the amino acids in one or another of the zinc fingers interfere with binding to the DNA of the response element.

### Renin

One of the functions of the kidney is to monitor blood pressure and take corrective action if it should drop. The kidney does this by secreting the proteolytic enzyme renin. Renin acts on angiotensinogen, a plasma peptide, splitting off a fragment containing 10 amino acids called angiotensin I. Angiotensin I is cleaved by a peptidase secreted by blood vessels called angiotensin converting enzyme (ACE)—producing angiotensin II, which contains 8 amino acids. Angiotensin II constricts the walls of arterioles closing down capillary beds; stimulates the proximal tubules in the kidney to reabsorb sodium ions; stimulates the adrenal cortex to release aldosterone. Aldosterone causes the kidneys to reclaim still more sodium and thus water increases the strength of the heartbeat; stimulates the pituitary to release the antidiuretic hormone (ADH, also known as arginine vasopressin). All of these actions lead to an increase in blood pressure.

### Skin

When ultraviolet radiation strikes the skin, it triggers the conversion of ehydrocholesterol (a cholesterol derivative) into calciferol (vitamin D₃). Calciferol travels in the blood to the liver where it is converted into 25[OH] vitamin D₃. This compound travels to the kidneys where it is converted into calcitriol (1,25 [OH]₂ vitamin D₃). This final step is promoted by the parathyroid hormone (PTH). Although called a vitamin, calciferol and its products fully qualify as hormones because they are made in certain cells, carried in the blood, affect gene transcription in target cells.

### Heart

### Natriuretic Peptides

In response to a rise in blood pressure, the heart releases two peptides: A-type Natriuretic Peptide (ANP). This hormone of 28 amino acids is released from stretched atria (hence the “A”).

### B-Type Natriuretic Peptide (BNP)

This hormone (29 amino acids) is released from the ventricles. (It was first discovered in brain tissue; hence the “B”.)

Both hormones lower blood pressure by relaxing arterioles inhibiting the secretion of renin and aldosterone inhibiting the reabsorption of sodium ions by the kidneys. The latter two effects reduce the reabsorption of water by the kidneys. So the volume of urine increases as does the amount of sodium excreted in it. The net effect of these actions is to reduce blood pressure by reducing the volume of blood in the circulatory system. These effects give ANP and BNP their name (natrium=sodium; uresis=urinate).

### Muscles

Animals use muscles to convert the chemical energy of ATP into mechanical work. Three different kinds of muscles are found in vertebrate animals. Heart muscle—also called cardiac muscle—makes up the wall of the heart. Throughout life, it contracts some 70 times per minute pumping about 5 liters of blood each minute. Smooth muscle is found in the walls of all the hollow organs of the body (except the heart). Its contraction reduces the size of these structures. Thus it regulates the flow of blood in the arteries moves your breakfast along through your gastrointestinal tract expels urine from your urinary bladder, sends babies out into the world from the uterus, and regulates the flow of air through the lungs. The contraction of smooth muscle is generally not under voluntary control. Skeletal muscle, as its name implies, is the muscle attached to the skeleton. It is also called striated muscle. The contraction of skeletal muscle is under voluntary control.

### Anatomy of Skeletal Muscle

A single skeletal muscle, such as the triceps muscle, is attached at its origin to a large area of bone; in this case, the humerus. At its other end, the insertion, it tapers into a glistening white tendon which, in this case, is attached to the ulna, one of the bones of the lower arm. As the triceps contracts, the insertion is pulled toward the origin and the arm is straightened or extended at the elbow. Thus the triceps is an extensor. Because skeletal muscle exerts force only when it contracts, a second muscle—a flexor—is needed to flex or bend the joint. The biceps muscle is the flexor of the lower arm. Together, the biceps and triceps make up an antagonistic pair of muscles. Similar pairs, working antagonistically across other joints, provide for almost all the movement of the skeleton.

### The Muscle Fiber

Skeletal muscle is made up of thousands of cylindrical muscle fibers often running all the way from origin to insertion. The fibers are bound together by connective tissue through which run blood vessels and nerves. Each muscle fibers contains: an array of myofibrils that are stacked lengthwise and run the entire length of the fiber (mitochondria, an extensive endoplasmic reticulum and many nuclei). The multiple nuclei arise from the fact that each muscle fiber develops from the fusion of many cells (called myoblasts). The number of fibers is probably fixed early in life. This is regulated by myostatin, a cytokine that is synthesized in muscle cells (and circulates as a hormone later in life). Myostatin suppresses skeletal muscle development. Cattle and mice with inactivating mutations in their myostatin genes develop much larger muscles. Some athletes and other remarkably strong people have been found to carry one mutant myostatin gene. These discoveries have already led to the growth of an illicit market in drugs supposedly able to suppress myostatin.

In adults, increased strength and muscle mass comes about through an increase in the thickness of the individual fibers and increase in the amount of connective tissue. In the mouse, at least, fibers increase in size by attracting more myoblasts to fuse with them. The fibers attract more myoblasts by releasing the cytokine interleukin 4 (IL-4). Anything that lowers the level of myostatin also leads to an increase in fiber size. Because a muscle fiber is not a single cell, its parts are often given special names such as sarcolemma for plasma membrane sarcoplasmic reticulum for endoplasmic reticulum, sarcosome for mitochondrion and sarcoplasm for cytoplasm, although this tends to obscure the essential similarity in structure and function of these structures and those found in other cells.

The nuclei and mitochondria are located just beneath the plasma membrane. The endoplasmic reticulum extends between the myofibrils. Seen from the side under the microscope, skeletal muscle fibers show a pattern of cross banding, which gives rise to the other name: striated muscle. The striated appearance of the muscle fiber is created by a pattern of alternating dark A bands and light I bands. The A bands are bisected by the H zone, the I bands are bisected by the Z line. Each myofibril is made up of arrays of parallel filaments. The thick filaments have a diameter of about 15 nm. They are composed of the protein myosin. The thin filaments have a diameter of about 5 nm. They are composed chiefly of the protein actin along with smaller amounts of two other proteins: troponin and tropomyosin.

The anatomy of a sarcomere. The thick filaments produce the dark A band. The thin filaments extend in each direction from the Z line. Where they do not overlap the thick filaments, they create the light I band. The H zone is that portion of the A band where the thick and thin filaments do not overlap.

The entire array of thick and thin filaments between the Z lines is called a sarcomere. Shortening of the sarcomeres in a myofibril produces the shortening of the myofibril and, in turn, of the muscle fiber of which it is a part.

### Activation of Skeletal Muscle

The contraction of skeletal muscle is controlled by the nervous system. In this respect, skeletal muscle differs from smooth and cardiac muscle. Both cardiac and smooth muscle can contract without being stimulated by the nervous system. Nerves of the autonomic branch of the nervous system lead to both smooth and cardiac muscle, but their effect is one of moderating the rate and/or strength of contraction.

### The Neuromuscular Junction

Nerve impulses (action potentials) traveling down the motor neurons of the sensory-somatic branch of the nervous system cause the skeletal muscle fibers at which they terminate to contract. The junction between the terminal of a motor neuron and a muscle fiber is called the neuromuscular junction. It is simply one kind of synapse. (The neuromuscular junction is also called the myoneural junction.) The terminals of motor axons contain thousands of vesicles filled with acetylcholine (ACh). When an action potential reaches the axon terminal, hundreds of these vesicles discharge their ACh onto a specialized area of postsynaptic membrane on the fiber. This area contains a cluster of transmembrane channels that are opened by ACh and let sodium ions (Na⁺) diffuse in.

The interior of a resting muscle fiber has a resting potential of about −95 mV. The influx of sodium ions reduces the charge, creating an end plate potential. If the end plate potential reaches the threshold voltage (approximately −50 mV), sodium ions flow in with a rush and an action potential is created in the fiber. The action potential sweeps down the length of the fiber just as it does in an axon. No visible change occurs in the muscle fiber during (and immediately following) the action potential. This period, called the latent period, lasts from 3-10 msec. Before the latent period is over, the enzyme acetylcholinesterase breaks down the ACh in the neuromuscular junction (at a speed of 25,000 molecules per second), the sodium channels close, and the field is cleared for the arrival of another nerve impulse. The resting potential of the fiber is restored by an outflow of potassium ions. The brief (1-2 msec) period needed to restore the resting potential is called the refractory period.

### Tetanus

The process of contracting takes some 50 msec; relaxation of the fiber takes another 50-100 msec. Because the refractory period is so much shorter than the time needed for contraction and relaxation, the fiber can be maintained in the contracted state so long as it is stimulated frequently enough (e.g., 50 stimuli per second). Such sustained contraction is called tetanus. When shocks are given at 1/sec, the muscle responds with a single twitch. At 5/sec and 10/sec, the individual twitches begin to fuse together, a phenomenon called clonus. At 50 shocks per second, the muscle goes into the smooth, sustained contraction of tetanus. Clonus and tetanus are possible because the refractory period is much briefer than the time needed to complete a cycle of contraction and relaxation. Note that the amount of contraction is greater in clonus and tetanus than in a single twitch. As we normally use our muscles, the individual fibers go into tetanus for brief periods rather than simply undergoing single twitches.

### The Sliding-Filament Model

Each molecule of myosin in the thick filaments contains a globular subunit called the myosin head. The myosin heads have binding sites for the actin molecules in the thin filaments and ATP. Activation of the muscle fiber causes the myosin heads to bind to actin. An allosteric change occurs which draws the thin filament a short distance (˜10 nm) past the thick filament. Then the linkages break (for which ATP is needed) and reform farther along the thin filament to repeat the process. As a result, the filaments are pulled past each other in a ratchetlike action. There is no shortening, thickening, or folding of the individual filaments. As a muscle contracts, the Z lines come closer together, the width of the I bands decreases, the width of the H zones decreases, but there is no change in the width of the A band. Conversely, as a muscle is stretched, the width of the I bands and H zones increases, but there is still no change in the width of the A band.

### Coupling Excitation to Contraction

Calcium ions (Ca²⁺) link action potentials in a muscle fiber to contraction. In resting muscle fibers, Ca²⁺ is stored in the endoplasmic (sarcoplasmic) reticulum. Spaced along the plasma membrane (sarcolemma) of the muscle fiber are inpocketings of the membrane that form tubules of the “T system”. These tubules plunge repeatedly into the interior of the fiber. The tubules of the T system terminate near the calcium-filled sacs of the sarcoplasmic reticulum. Each action potential created at the neuromuscular junction sweeps quickly along the sarcolemma and is carried into the T system.

The arrival of the action potential at the ends of the T system triggers the release of Ca²⁺. The Ca²⁺ diffuses among the thick and thin filaments where it binds to troponin on the thin filaments. This turns on the interaction between actin and myosin and the sarcomere contracts. Because of the speed of the action potential (milliseconds), the action potential arrives virtually simultaneously at the ends of all the tubules of the T system, ensuring that all sarcomeres contract in unison. When the process is over, the calcium is pumped back into the sarcoplasmic reticulum using a Ca²⁺ ATPase.

### Isotonic Versus Isometric Contractions

If a stimulated muscle is held so that it cannot shorten, it simply exerts tension. This is called an isometric (“same length”) contraction. If the muscle is allowed to shorten, the contraction is called isotonic (“same tension”).

### Motor Units

All motor neurons leading to skeletal muscles have branching axons, each of which terminates in a neuromuscular junction with a single muscle fiber. Nerve impulses passing down a single motor neuron will thus trigger contraction in all the muscle fibers at which the branches of that neuron terminate. This minimum unit of contraction is called the motor unit. The size of the motor unit is small in muscles over which we have precise control. Examples: a single motor neuron triggers fewer than 10 fibers in the muscles controlling eye movements, the motor units of the muscles controlling the larynx are as small as 2-3 fibers per motor neuron. In contrast, a single motor unit for a muscle like the gastrocnemius (calf) muscle may include 1000-2000 fibers (scattered uniformly through the muscle). Although the response of a motor unit is all-or-none, the strength of the response of the entire muscle is determined by the number of motor units activated. Even at rest, most of our skeletal muscles are in a state of partial contraction called tonus. Tonus is maintained by the activation of a few motor units at all times even in resting muscle. As one set of motor units relaxes, another set takes over.

### Fueling Muscle Contraction

ATP is the immediate source of energy for muscle contraction. Although a muscle fiber contains only enough ATP to power a few twitches, its ATP “pool” is replenished as needed. There are three sources of high-energy phosphate to keep the ATP pool filled: creatine phosphate, glycogen and cellular respiration in the mitochondria of the fibers.

### Creatine Phosphate

The phosphate group in creatine phosphate is attached by a “high-energy” bond like that in ATP. Creatine phosphate derives its high-energy phosphate from ATP and can donate it back to ADP to form ATP.

Creatine phosphate+ADP [symbol] creatine+ATP

The pool of creatine phosphate in the fiber is about 10 times larger than that of ATP and thus serves as a modest reservoir of ATP.

### Glycogen

Skeletal muscle fibers contain about 1% glycogen. The muscle fiber can degrade this glycogen by glycogenolysis producing glucose-1-phosphate. This enters the glycolytic pathway to yield two molecules of ATP for each pair of lactic acid molecules produced. Not much, but enough to keep the muscle functioning if it fails to receive sufficient oxygen to meet its ATP needs by respiration.

However, this source is limited and eventually the muscle must depend on cellular respiration.

### Cellular Respiration.

Cellular respiration not only is required to meet the ATP needs of a muscle engaged in prolonged activity (thus causing more rapid and deeper breathing), but is also required afterwards to enable the body to resynthesize glycogen from the lactic acid produced earlier (deep breathing continues for a time after exercise is stopped). The body must repay its oxygen debt.

Type I Vs. Type II Fibers

Two different types of muscle fiber can be found in most skeletal muscles. The Type I and Type II fibers differ in their structure and biochemistry.

Type I Fibers are loaded with mitochondria and depend on cellular respiration for ATP production, resistant to fatigue, rich in myoglobin and hence red in color, activated by small-diameter, thus slow-conducting, motor neurons, also known as “slow-twitch” fibers and dominant in muscles that depend on tonus, e.g., those responsible for posture.

Type II Fibers are few mitochondria, rich in glycogen and depend on glycolysis for ATP production, fatigue easily, low in myoglobin hence whitish in color, activated by large-diameter, thus fast-conducting, motor neurons, also known as “fast-twitch” fibers and dominant in muscles used for rapid movement.

Most skeletal muscles contain some mixture of Type I and Type II fibers, but a single motor unit always contains one type or the other, never both.

### Cardiac Muscle

Cardiac or heart muscle resembles skeletal muscle in some ways: it is striated and each cell contains sarcomeres with sliding filaments of actin and myosin. However, cardiac muscle has a number of unique features that reflect its function of pumping blood. The myofibrils of each cell (and cardiac muscle is made of single cells—each with a single nucleus) are branched. The branches interlock with those of adjacent fibers by adherens junctions. These strong junctions enable the heart to contract forcefully without ripping the fibers apart. The action potential that triggers the heartbeat is generated within the heart itself. Motor nerves (of the autonomic nervous system) do run to the heart, but their effect is simply to modulate—increase or decrease—the intrinsic rate and the strength of the heartbeat. Even if the nerves are destroyed (as they are in a transplanted heart), the heart continues to beat.

The action potential that drives contraction of the heart passes from fiber to fiber through gap junctions. Significance: All the fibers contract in a synchronous wave that sweeps from the atria down through the ventricles and pumps blood out of the heart. Anything that interferes with this synchronous wave (such as damage to part of the heart muscle from a heart attack) may cause the fibers of the heart to beat at random—called fibrillation. Fibrillation is the ultimate cause of most deaths and its reversal is the function of defibrillators that are part of the equipment in ambulances, hospital emergency rooms, and—recently—even on U.S. air lines. The refractory period in heart muscle is longer than the period it takes for the muscle to contract (systole) and relax (diastole). Thus tetanus is not possible (a good thing, too!). Cardiac muscle has a much richer supply of mitochondria than skeletal muscle. This reflects its greater dependence on cellular respiration for ATP. Cardiac muscle has little glycogen and gets little benefit from glycolysis when the supply of oxygen is limited. Thus anything that interrupts the flow of oxygenated blood to the heart leads quickly to damage—even death—of the affected part. This is what happens in heart attacks.

### Smooth Muscle

Smooth muscle is made of single, spindle-shaped cells. It gets its name because no striations are visible in them. Nonetheless, each smooth muscle cell contains thick (myosin) and thin (actin) filaments that slide against each other to produce contraction of the cell. The thick and thin filaments are anchored near the plasma membrane (with the help of intermediate filaments). Smooth muscle (like cardiac muscle) does not depend on motor neurons to be stimulated. However, motor neurons (of the autonomic system) reach smooth muscle and can stimulate it—or relax it—depending on the neurotransmitter they release (e.g. noradrenaline or nitric oxide, NO)). Smooth muscle can also be made to contract by other substances released in the vicinity (paracrine stimulation). Example: release of histamine causes contraction of the smooth muscle lining our air passages (triggering an attack of asthma) by hormones circulating in the blood. Example: oxytocin reaching the uterus stimulates it to contract to begin childbirth. The contraction of smooth muscle tends to be slower than that of striated muscle. It also is often sustained for long periods. This, too, is called tonus but the mechanism is not like that in skeletal muscle.

### Muscle Diseases

### The Muscular Dystrophies (MD)

Together myosin, actin, tropomyosin, and troponin make up over three-quarters of the protein in muscle fibers. Some two dozen other proteins make up the rest. These serve such functions as attaching and organizing the filaments in the sarcomere and connecting the sarcomeres to the plasma membrane and the extracellular matrix. Mutations in the genes encoding these proteins may produce defective proteins and resulting defects in the muscles. Among the most common of the muscular dystrophies are those caused by mutations in the gene for dystrophin.

The gene for dystrophin is huge, containing 79 exons spread out over 2.3 million base pairs of DNA. Thus this single gene represents about 0.1% of the entire human genome (3×10⁹ bp) and is almost half the size of the entire genome of E. coli!

### Duchenne Muscular Dystrophy (DMD)

Perhaps its great size makes this gene so susceptible to partial deletions. If these cause a change in the reading frame, no dystrophin is synthesized and DMD, a very severe form of the disease, results.

### Becker Muscular Dystrophy (BMD).

If the deletion simply removes certain exons, a shortened protein results that produces BMD, a milder form of the disease. The gene for dystrophin is on the X chromosome, so these two diseases strike males in a typical X-linked pattern of inheritance.

### Myasthenia Gravis

Myasthenia gravis is an autoimmune disorder affecting the neuromuscular junction. Patients have smaller end plate potentials (EPPs) than normal. With repeated stimulation, the EPPs become too small to trigger further action potentials and the fiber ceases to contract. Administration of an inhibitor of acetylcholinesterase temporarily can restore contractility by allowing more ACh to remain at the site. Patients with myasthenia gravis have only 20% or so of the number of ACh receptors found in normal neuromuscular junctions. This loss appears to be caused by antibodies directed against the receptors. Some evidence: A disease resembling myasthenia gravis can be induced in experimental animals by immunizing them with purified ACh receptors; Anti-ACh receptor antibodies are found in the serum of human patients; Experimental animals injected with serum from human patients develop the signs of myasthenia gravis. Newborns of mothers with myasthenia gravis often show mild signs of the disease for a short time after their birth. This is the result of the transfer of the mother's antibodies across the placenta during gestation. The reason some people develop autoimmune antibodies against the ACh receptor is unknown.

### The Cardiac Myopathies

Cardiac muscle, like skeletal muscle, contains many proteins in addition to actin and myosin. Mutations in the genes for these may cause the wall of the heart to become weakened and, in due course, enlarged. Among the genes that have been implicated in these diseases are those encoding: actin, two types of myosin, troponin, tropomyosin, myosin-binding protein C (which links myosin to titin). The severity of the disease varies with the particular mutation causing it (over 100 have been identified so far). Some mutations are sufficiently dangerous that they can lead to sudden catastrophic heart failure in seemingly healthy and active young adults.

### Hormones of the Liver

The liver synthesizes and secretes at least three important hormones: Insulin-like Growth Factor-1 (IGF-1), Angiotensinogen, Thrombopoietin.

### Insulin-Like Growth Factor-1

This protein of 70 amino acids was once called somatomedin because it, not growth hormone, is the immediate stimulus for growth of the body. Growth hormone released from the anterior lobe of the pituitary binds to receptors on the surface of liver cells which stimulates the synthesis and release of IGF-1 from them. Many cells have receptors for IGF-1, especially cells in the bone marrow and in the cartilaginous growing regions of the long bones. Binding of IGF-1 to cells with receptors for it stimulates them to move from G₁ of the cell cycle to S phase and on to mitosis.

Mice with one of their Igf-1 receptor genes “knocked out” live 25% longer than normal mice. This may result from an increase in their resistance to the damaging effects of reactive oxygen species (ROS). These heterozygous mice appear to be normal in every other respect. The levels of IGF-1 in the blood are highest during the years of puberty which is, of course, a time of rapid growth. Occasionally children are found that have stunted growth because they have inherited mutant genes for the growth hormone (GH) receptor. Recombinant human IGF-1 has been successfully used to treat them.

### Angiotensinogen

This protein is released into the blood where it serves as the precursor for angiotensin. How angiotensin is manufactured, and the role it plays in maintaining blood pressure is described in the discussion of renin.

### Thrombopoietin (TPO)

Thrombopoietin is a protein of 332 amino acids. It stimulates precursor cells in the bone marrow to differentiate into megakaryocytes. Megakaryocytes generate platelets, essential to blood clotting. The production of megakaryocytes—and thus platelets—is under homeostatic control. It works like this: Circulating platelets are covered with receptors for TPO. So are megakaryocytes and their precursors, but there are fewer of them. When platelet counts are high, most of the circulating TPO is bound to the platelets and less is left to stimulate megakaryocytes. When platelet counts drop, more TPO becomes available to stimulate megakaryocytes to replenish the platelet supply. A segment of thrombopoietin, manufactured by recombinant DNA technology, is now available for human therapy. It already shows promise in quickly restoring normal platelet counts in patients who have undergone chemotherapy.

Because the inventor herein has found that the present invention is significantly beneficial for the treatment of worms in animals such as dogs, cats, horses and cattle, the follow is background on current chemical based de-wormers. Listed blelow are descriptions of different chemical clases of equine dewormers. Each described how that chemical class affects parasites and which brand names are associated with it. All of the molecules within the same chemical class work in similar ways. Therefore, it is critical that you rotate not only between brand names, but between chemical classes in order to create a truly effective deworming program for your horse. This practice will help reduce the chances of developed resistance in parasites, as well as to maximize the best attributes of each chemical compound. BENZIMIDAZOLES: Kills parasites quickly and offers broad spectrum nematode protection, including large stongyles, small strongyles, ascarids and pinworms. Brand Names: Antheleide EQ (oxibendazole), Panacur Powerpac (fenbendazole), Safe-Guard (fenbendazole), Benzelmin (oxfendazole). PYRANTELS: Kill parasites slowly by causing paralysis in a broad spectrum of nematodes, such as large and small strongyles, ascarids and pinworms. Brand Names: Strongid Paste (pyrantel pamoate), Strongid C (pyrantel tartrate), Strongid C2X (pyrantel tartrate), Continuex Daily Dewormer (pyrantel tartrate), Rotation 2 (pyrantel pamoate), Equi Aid CW (pyrantel tartrate), Strongyle Wormer (pyrantel tartrate), Manna Pro Foal & Horse Pelleted Wormer (pyrantel tartrate), Nu-Image Guardian (pyrantel tartrate). MACROCYCLIC LACTONES: Causes paralysis in parasites, including a broad spectrum of nematodes and arthropods. It affects large and small strongyles, benzimidazole-resistant small strongyles, ascarids, pinworms, bots and summer sore-causing parasites. Brand Names: Equell (ivermectin), Zimercterin (ivermectin), IverCare (ivermectin). Quest Gel (moxidectin), Eqvalan (ivermectin), Equimectrin (ivermectin), Rotation 1 (ivermectin), Horse Health Equine Ivermectin (ivermectin), Agri-Mectin Equine Paste (ivermectin). COMBINED MACROCYCLIC LACTONES: Paralyzes nematodes and arthropods and breaks down the membranes of tapeworms. This class protects against large and small stongyles, benzimidazole-resistant small strongyles, ascarids, pinworms, bots, summer sore-causing parasites and tapeworms. Brand Names: Equimax (ivermectin/praziquantel), Zimectrin Gold (ivermectin/praziquantel), Quest jPlus (moxidectin/praziquantel), ComboCare (moxidectin/praziquantel).

### SUMMARY OF THE INVENTION

The present invention relates to a discovery that a mastic gum herb alone, or as an extract thereof, or as a 4 to 1 standardized extract thereof, or combined with minerals and/or trace minerals is highly beneficial when taken as a dietary supplement/hernal agent or formula for nutritional benefits, and have surprising efficacy in a nutritional dietary herb for the support structure and function, maintaining, increasing or lowering and production, as a prescription or over the counter drug, or as an injectable to prevent or cure diseases and/or treatment of the kidneys which relate to various conditions including: problems of blood perishability.

The kidney secretes two hormones: Erythropoietin (EPO) and Calcitriol (1,25[OH]₂ Vitamin D₃) as well as the enzyme renin. Erythropoietin (EPO) is a glycoprotein. It acts on the bone marrow to increase the production of red blood cells.

By taking Mastic gum alone, or as a 4 to 1 standardized extract thereof, or combined with minerals and/or trace minerals, the invention helps support the structure and function of the kidneys which in turn helps the kidney secrete two hormones: Erythropoietin (EPO) and Calcitriol (1,25[OH]₂ Vitamin D₃), as well as the enzyme renin. The invention further helps support/treat hematocrit hemoglobin, red white blood cell type anemia and HIV Aids.

Magnesium is predominately an intracellularcation, the effectiveness of the oral supplement is assessed by its solubility and rate of uptake from the small intestine into the bloodstream and by its transfer into the tissues. Magnesium balance is regulated by the kidneys (White et al., 1992).

Because the invention is primarily directed at supporting and improving the performance of the kidneys, as a secondary benefit, the invention is beneficial for the hormones of the kidneys, skin, heart, fibrinogen, blood clots, all blood cells including red and white blood cell types and functions, cancer cells, PH balance, acidic blood, anemia, septicemia, hemoglobin, Erythropoietin (EPO, glycoprotien), hematocrit, liver, lactic acid, oxygen to muscles, skeletal muscles, bone, calcitriol (1,25(OH)2 vitamin D3), rickets, sicle cell anemia, immune system enzyme renin, urea protein and cycle, proteins and other macromolecules, colostrum, glucose, ornithine transcarbamoylase, Bowman's capsule, oxygen transport, carbon dioxide transport, ammonia, amino acids, reduction in heart rate, stroke, allergies, enzymes, AST—Aspartate Transaminase enzymes: AST, Alkaline Phosphotase, CPK, GGT, and the chemicals Bilirubin, blood urea nitrogen (BUN), Urea and Creatinine, poor appetite, and muscle tie-up, for humans and animals such as horses, cattle, dogs and cats. Other useful areas where the invention is of beneficial use is urinary incontinence in children as well as in women and men, lupus nephritis, high or low blood pressure, diabetes type 1 or type 2, homocysteine, proteinuria glomerulonephritis, or simply nephritis, kidney stones, calcium struvite uric acid stones cystine stones, male reproductive system, female reproductive system, milk urea nitrogen concentration: heritability and genetic correlations with reproductive performance and disease, common colds, influenza, Herpe's virus, E-Coli bacteria and I.B.S.

The invention has also been found to be extremely beneficial for the treatment of worms (de-worming) in animals such as horses, cows, cats and dogs.

As mentioned above, the present invention is related generally to a dietary supplement and/or herbal formula and/or herbal agent to be used primarily as a structure and/or function or support and/or treatment of the kidneys, wherein various medical conditions can be improved and treated. Medical conditions and/or organs, for which the invention is beneficial include hormones of the kidneys, low Hematocrit, Low Hemoglobin, increase erythropoietin, liver, low Ph, muscle Azoturia and muscle tie-Up syndrome, Anemia, Septicemia, Metabolic Acidosis, Joints, ligaments, Tendons, excessive Ammonia and Carbon Dioxide, low colostrum, reabsorbing of Glucose, Amino Acids, and Lactic acid, Gout, Irritable Bowel Syndrome, Protein deamination in the liver, decrease in GFR, Renal Calculi, Urinary tract infections, Glomerular disease, renal failure, Polycystic Kidney disease, kidney stones, weakened bones, Osteomalcia, oxygen to the brain, stroke, Rickets, Decrease of Appetite, and muscle tie-up, among other conditions.

By taking Mastic gum alone, or as a 4 to 1 standardized extract thereof, or as the extract combined with minerals and/or trace minerals, the invention helps remove ammonia toxic mineral metal and protein built up from the body liver and kidneys preventing harming the cells and causing kidney stones. Urea is a nitrogenous product made by the liver. Nitrogenous wastes and ammonia from ammonia producing bacteria (urease enzyme) are initially brought to the liver as ammonia, a chemical compound of nitrogen so toxic that it could not remain in the body without harming healthy cells. The liver removes ammonia from the blood and converts it to the less harmful substance urea. The urea enters the bloodstream and is then removed by the kidneys. The underlying basis for the invention is that the invention effectively improves the performance of the kidneys so that the kidneys can properly process the waste. In turn, because the kidneys performance is improved, the aforementioned medical conditions can be addressed and the performance of the associated organs can be improved. This is the type of benefit that enhances the invention's capability to proliferate healthy cells and kills or inhibits growth or development of cancer cells or other diseases.

### DESCRIPTION OF THE PREFERRED EMBODIMENTS

The disclosed embodiments of the present invention display a certain preferred composition but are not intended to limit the scope of the invention. Although mastic gum can be administered by itself as an extract, it is preferred that it be administered as a composition so as to be ingested in the form of a liquid, capsule (pill, tablet), or mixed with a meal to be eating by the subject. Alternatives are the use of syringes with water or in the form of a paste.

The botanical name for the mastic-tree gum is Pistacia Lentiscus.

By way of example only, if the invention were to be administered to a human, then a capsule form may include 150 milligrams of a mastic gum herb and/or extract of the herb. Optionally, active ingredients such as minerals may be included, for example, 125 milligrams of various minerals and 250 milligrams of magnesium and a salt thereof such as magnesium sulfate. Minerals may include coral, calcium and a salt or ion thereof such as coral calcium, in the form of a powder or liquid. Trace minerals that may be included in such coral calcium are sulfur, chloride, lead, aluminum, antimony, arsenic, barium, beryllium, bismuth boron bromine cadmium, cesium chromium cobalt copper, mercury, molybdenum nickel, carbon, fluoride, iodine, selenium, vanadium and a wide variety of other trace minerals, none of which are of a concentration to be harmful to the subjects.

By way of example for animals or humans, a serving may include from about 10 micro-grams to about 20,000 milligrams of mastic gum herb and/or extract thereof. Minerals may be added, for example, about 10 micro-grams to about 20,000 milligrams of minerals and trace minerals and/or their salts may be added.

The inventor herein discovered that by using about 10 micro-grams to about 20,000 milligrams of Mastic Gum from the Pistacia Lentiscus tree, and optionally 10 micro-grams milligram to about 20,000 milligram of one to one hundred and fifty minerals and trace minerals, whether organic, inorganic or ionic form, and other elements and excipients, the above-mentioned conditions in humans and animals can be effectively treated. Particularly the minerals from the Dead Sea of Israel, the Great Salt Lake of Utah, or the Coral from the oceans is preferred source of minerals. About 10 micro-grams to about 20,000 milligrams of magnesium may also be optionally added to the formulation. It is highly beneficial when put into a capsule, tablet, liquid, or powder form, for humans, horses, cattle, dogs, or cats, and taken once or more times a day, or administered to a horse in its feed or one may add two ounces of water or paste in a syringe with the formula and syringed in back of the mouth to the throat area.

The inventor herein also discovered that by adding about 10 micrograms to about 5000 milligrams of Vanadium in a number of animal and a few human studies, vanadyl sulfate has improved insulin sensitivity and reduced blood sugar in those with both type 1 and type 2 diabetes. In one study of people with type 2 diabetes, vanadium also lowered their total and LDL (“bad”) cholesterol.

Although these studies show promise, the long-term safety of vanadium has not been established. For example, the use of vanadium is not advised because of the potential toxic effects associated with high doses of this mineral to the kidneys. Vanadyl sulfate helps improve high blood pressure.

### Bipolar Disorder (Manic/Depression)

Vanadium levels may be elevated during manic episodes and blood levels may be high during times of depression. This is particularly true if the mood disorder is accompanied by psychosis (particularly delusional thoughts). Vanadium would be helpful for people with bipolar disorder.

Recent and on-going research has found that vanadium has a benefit in that it kills cancer cells.

Vanadium exists in several forms, including vanadyl sulfate and vanadate. Vanadyl sulfate is most commonly found in nutritional supplements. Because of its potential toxicity, some experts believe that vanadium should be considered a drug and not a nutritional supplement.

What the inventor has found is that by adding vanadium with about 10 mcg to about 20000 mg of Mastic Gum by itself or as a 4 to 1 standardized extract, there were no signs of toxity to the liver or kidneys. The use therefore of Mastic Gum with the vanadium may be a very effective treatment for combating cancer.

The inventor herein discovered that by formulating 10 micro-grams to 20000 milligrams of magnesium and its salts and about 10 mcg to about 20000 mg of Mastic gum by itself or as a 4 to 1 standardized extract has improved magnesium absorption and bioavailability. Magnesium is absorbed primarily in the distal small intestine, and healthy people absorb approximately 30% to 40% of ingested magnesium. Since magnesium is predominately an intracellularcation, the effectiveness of the oral supplement is assessed by its solubility and rate of uptake from the small intestine into the bloodstream and by its transfer into the tissues. Magnesium balance is regulated by the kidneys.

### Improving Magnesium Absorption and Bioavailability

Various lines of research have established a connection between hypomagnesaemia and an extensive inventory of disease states. Because the signs and symptoms of hypomagnesaemia may be indistinct from those of other conditions, the deficiency is often difficult to identify clinically. Moreover, body magnesium may be deficient even when serum values are normal, and the deficiency may be specific to a particular organ. Studies have shown that between 6.9% and 11% of hospitalized patients and 65% of patients in intensive care units may have magnesium deficiency.

### Specific Disease Effects

Cardiovascular diseases such as heart failure, cardiac dysrhythmia and hypertension, lead the list of disorders associated with hypomagnesemia. The relation of serum and dietary magnesium with coronary heart disease (CHD) incidence was examined in 13,922 middle-aged adults from four U.S. communities (Liao et al., 1998). Over four to seven years of follow-up, CHD developed in 223 men and 96 women. After adjusting for sociodemographic characteristics, waist/hip ratio, smoking, alcohol consumption, sports participation, use of diuretics, fibrinogen, total and high-density lipoprotein cholesterol levels, triglyceride levels, and hormone replacement therapy, the researchers concluded that magnesium deficiency has the potential to contribute to the pathogenesis of coronary atherosclerosis or acute thrombosis.

A randomized, double-blind, placebo-controlled trial in an acute-care hospital was conducted to determine whether magnesium administration would reduce morbidity and mortality after cardiac surgery (England et al., 1992). Over a six-month period, 100 patients electively scheduled for cardiac surgery involving cardiopulmonary bypass were studied. Fifty patients received an intravenous infusion of a magnesium supplement and 50 patients received a placebo after the termination of cardiopulmonary bypass. The magnesium-treated patients had a significantly decreased frequency of postoperative ventricular dysrhythmias compared to placebo-treated patients (p<0.04). Magnesium-treated patients also had significantly higher postoperative cardiac indices in the intensive care unit (p<0.02).

The effects of magnesium supplementation on office, home and ambulatory blood pressures were studied in 60 untreated or treated hypertensive Japanese patients (34 men and 26 women, aged 33 to 74 years) with office blood pressure >140/90 mmHg (Kawano et al., 1998). The patients were assigned to an eight-week magnesium supplementation period or an eight-week control period in a randomized crossover design. Magnesium supplementation lowered blood pressure in hypertensive patients, and this effect was greater in those with higher blood pressure. The results supported the usefulness of increasing magnesium intake as a lifestyle modification in the management of hypertension.

Hypomagnesaemia has also been linked to chronic fatigue syndrome (CFS). The hypothesis that patients with CFS have low levels of red blood cell magnesium was tested in a British case-control study (Cox et al., 1991). In this study, patients with CFS had lower red cell magnesium concentrations than did healthy control subjects matched for age, sex and social class. In the clinical trial, patients with CFS were randomly given a magnesium supplement (n=15) or placebo (n=17). Patients treated with magnesium claimed to have improved energy levels, a better emotional state and less pain as judged by changes in the Nottingham health profile.

According to a 1998 review by Mauskop and Altura, “The importance of magnesium in the pathogenesis of migraine headaches has been clearly established by a large number of clinical and experimental studies.” The exact role of low magnesium levels in migraine development is unknown, but magnesium concentration affects serotonin receptors, nitric oxide synthesis and release, N-methyl-D-aspartate (NMDA) receptors, and other migraine-related receptors and neurotransmitters. According to this review, as much as 50% of patients have lowered levels of ionized magnesium during an acute migraine attack. In these patients, an infusion of magnesium results in a rapid and sustained relief of an acute migraine (Mauskop and Altura, 1998).

It is probable that some conditions, such as CFS and migraine, are related to catecholamine release and a “spurious hypomagnesemia” as opposed to low magnesium levels per se. However, hypomagnesemia is a common problem in hyperthyroid patients. This is of particular concern in the elderly with their predilection to develop atrial fibrillation. In fact, alcoholics are probably the largest population at risk for hypomagnesemia as well as a whole host of other metabolic derangements.

Magnesium deficiency in conjunction with diabetes also has the potential to intensify some complications associated with the disease. A study of 23 children with diabetes found that their serum values of total and ionized calcium, magnesium, intact parathyroid hormone, calcitriol, and osteocalcin were lower than those of control subjects (Saggese et al., 1991). All patients were given 6 mg/kg daily (orally) of elemental magnesium for up to 60 days. During treatment, all concentrations increased significantly, reaching control values. These data suggested that magnesium deficiency plays a pivotal role in positively altering mineral homeostasis in insulin-dependent diabetes mellitus.

Poor nutritional status, impaired gastrointestinal (GI) status and polypharmacy are other factors that have been found to put the elderly at greater risk for magnesium deficiency. Therapy with thiazide or loop diuretics for hypertension or congestive heart failure, and the consequent diuresis, may further stress their mineral balance. Hypokalemia can be a problem in the elderly population, and Klein (1994) reported finding hypomagnesemia in 38% to 42% of hypokalemic patients. Because the correction of a potassium deficit may be difficult to achieve unless the magnesium deficit is also corrected, patients with hypokalemia should also be evaluated for magnesium deficiency. The elderly have also been found to vulnerable to hypomagnesemia-induced malabsorption syndromes and nephrolithiasis.

### Correcting the Problem

The optimal daily intake of magnesium for an adult is typically 15 mmol to 20 mmol (30 mEq to 40 mEq), and normal magnesium serum levels range from 0.7 mmol/L to 1.0 mmol/L. Foods that are rich in magnesium include legumes, whole grains, green leafy vegetables, nuts, coffee, chocolate and milk. Although these foods are readily available, some individuals do not consume adequate quantities to satisfy the daily nutritional requirement. Furthermore, expanded consumption of processed foods, which tend to contain less magnesium, may account for the perceptible decline in dietary magnesium in the United States during the past century. Thus, continued use of an oral magnesium supplement that offers reliable absorption and bioavailability is recommended for people with magnesium deficiency. Oral magnesium supplements are available in a number of formulations that utilize a different anion or salt—such as oxide, gluconate, chloride or lactate dihydrate. However, these preparations are not interchangeable because they have differences in absorption and bioavailability.

Magnesium is absorbed primarily in the distal small intestine, and healthy people absorb approximately 30% to 40% of ingested magnesium. Since magnesium is predominately an intracellularcation, the effectiveness of the oral supplement is assessed by its solubility and rate of uptake from the small intestine into the bloodstream and by its transfer into the tissues. Magnesium balance is regulated by the kidneys. When magnesium levels in the blood are high, the kidneys will rapidly excrete the surplus. When magnesium intake is low, on the other hand, renal excretion drops to 0.5 mmol to 1 mmol (1 mEq to 2 mEq) per day. A caveat: patients with renal failure receiving magnesium salts need to be carefully monitored for the potential of magnesium intoxication.

### Magnesium Salts

The in vitro solubility and in vivo GI absorbability of magnesium oxide and magnesium citrate were compared (Lindberg et al., 1990). The simulated gastric fluids represented five different concentrations of hydrochloric acid. Magnesium citrate was significantly more soluble than magnesium oxide in all levels of acid secretion, but reprecipitation from magnesium oxide and magnesium citrate did not occur when the hydrochloric acid was titrated to a pH between 6 and 7, which is the pH of the distal small intestine where magnesium anions are absorbed. Absorption of the two magnesium formulations was also compared in vivo by measuring the rise in urinary magnesium levels, and the citrate form was absorbed to a much greater extent than the oxide.

The study just described involved healthy individuals with normal magnesium serum levels. In contrast, another study focused on the effects of magnesium supplementation in 40 elderly magnesium-deficient patients and compared oral versus intravenous administration (Gullestad et al., 1991-1992). The oral magnesium lactate-citrate preparation was given for six weeks at a daily dose of 15 mmol; the IV magnesium sulfate formulation was given at a daily dose of 30 mmol as an infusion in 1000 mL of saline for seven days. The two routes of magnesium administration yielded comparable results. The authors termed bioavailability of oral magnesium lactate citrate “satisfactory” and concluded that oral delivery of magnesium supplements for six weeks may restore magnesium levels in magnesium-deficient patients.

A non-randomized clinical trial evaluated the absorption of sustained-release magnesium lactate dihydrate in 24 patients (Kann, 1989). The patients received 21 mEq of the sustained-release preparation at 8 a.m. and 2 p.m. on the third day of the study after consuming a low-magnesium diet for two days. Blood samples were collected on day 2 and after the initial dose (day 3), and urine was collected for four continuous days. Statistical data showed that the participants absorbed 41% of the oral dose with no serious adverse reactions. In a study with dogs, magnesium L-lactate dihydrate proved to be highly soluble at a neutral pH with a readily absorbed anion, and decreased acidity did not impair its bioavailability (Robbins et al., 1989).

In conclusion, magnesium deficiency has been linked to a growing number of disease states. When hypomagnesemia is detected, the appropriate course of action consists of addressing the underlying cause (if identifiable) and reversing the depleted state. Oral magnesium supplements constitute an effective form of replacement therapy, but not all formulations are equal. Absorption and bioavailability of preparations vary, as do concomitant side effects. Various investigators have reported that magnesium L-lactate dihydrate, which is available in a sustained-release formulation, ensures maximal absorption in the distal small intestine. The solubility and bioavailability of magnesium L-lactate dihydrate are higher than those of other magnesium formulations, and the low incidence of side effects and a bid dosing schedule may provide the additional benefit of patient compliance.

References that discussed the above studies related to magnesium are: Cox IM, Campbell M J, Dowson D (1991), Red blood cell magnesium and chronic fatigue syndrome, Lancet 337(8744):757-760 [see comments]; England M R, Gordon G, Salem M, Chernow B (1992), Magnesium administration and dysrhythmias after cardiac surgery, A placebo-controlled, double-blind, randomized trial. JAMA 268(17): 2395-2402 [see comment]; Gullestad L, Oystein Dolva L, Birkeland K et al. (1991-1992), Oral versus intravenous magnesium supplementation in patients with magnesium deficiency. Magnes Trace Elem 10(1): 11-16; Kann J (1989), Absorption Study of Sustained Release Magnesium L-Lactate Dihydrate. Pittsburgh: Biodecisions Laboratories; Kawano Y, Matsuoka H, Takishita S, Omae T (1998), Effects of magnesium supplementation in hypertensive patients: assessment by office, home, and ambulatory blood pressures. Hypertension 32(2):260-265; Klein M (1994), Magnesium therapy in cardiovascular disease, Cardiovasc Rev Rep 15(10):9-56; Knochel J P (1991), Disorders of magnesium metabolism. In: Harrison's Principles of Internal Medicine, 12th ed., Wilson J D, Braunwald E, Isselbacher K J et al., eds. New York: McGraw-Hill Inc, pp 1935-1938; Liao F, Folsom A R, Brancati F L (1998), Is low magnesium concentration a risk factor for coronary heart disease? The Atherosclerosis Risk in the Communities (ARIC) Study. Am Heart J 136(3):480-490; Lindberg J S, Zobitz M M, Poindexter J R, Pak C Y C (1990), Magnesium bioavailability from magnesium citrate and magnesium oxide. J Am Coll Nutr 9(1):48-55; Mauskop A, Altura B M (1998), Role of magnesium in the pathogenesis and treatment of migraines. Clin Neurosci 5(1):24-27; Robbins T L, Imondi A R, Murphy P E et al. (1989), Magnesium lactate bioavailability in dogs is not impaired by decreased gastric acidity. J Am Coll Nutr 8:462. Abstract 144; Saggese G, Federico G, Bertelloni S et al. (1991), Hypomagnesemia and the parathyroid hormone-vitamin D endocrine system in children with insulin-dependent diabetes mellitus: effects of magnesium administration. J Pediatr 118(2):220-225 [see comment]; and White J, Massey L, Gales S K et al. (1992), Blood and urinary magnesium kinetics after oral magnesium supplements. Clin Ther 14(5):678-687.

Some of the conditions magnesium may be useful in treating or preventing are: aging, aggressive behavior, alcoholism, amytrophic lateral sclerosis, alzheimer's disease, arrhythmia, asthma, attention deficit disorder, autism, brain damage, cancer, cerebral palsy, cerebrovascular, chemical sensitivity, chronic fatigue, cluster headaches, cocaine-related stroke, constipation, cramps, diabetes, fibromyalgia, fluoride toxicity, head injuries, central nervous system injuries, heart disease, heart attack, atherosclerosis, cardiovascular disease, etc., HIV, AIDS, hypertension, kidney stones, magnesium deficiency, menopause, migraine headache, mitral valve prolapse, multiple sclerosis, nystagmus, osteoporosis, peripheral vascular disease, pregnancy-related problems, eclampsia, premenstrual syndrome (PMS), psychiatric disorders, repetitive strain injury, rrheumatoid arthritis, sickle cell disease, SIDS, sports-related problems, stress, stuttering, tetanus, tinnitis, sound sensitivity, TMJ, toxic shock, violence.

As mentioned above, the invention can be used for the support, function, and/or treatment of blood urea nitrogen (BUN) (a breakdown product of protein metabolism) in the blood. The BUN test is a somewhat routine test used primarily to evaluate renal (kidney) function. The test is often performed on patients with many different diseases.

Urea is formed in the liver as the end product of protein metabolism. During digestion, protein is broken down to amino acids. Amino acids contain nitrogen, which is removed as NH4+ (ammonium ion), while the rest of the molecule is used to produce energy or other substances needed by the cell. The ammonia is combined with other small molecules to produce urea. The urea makes its way into the blood and it is ultimately eliminated in the urine by the kidneys. Most renal diseases affect urea excretion so that BUN levels increase in the blood. Patients with dehydration or bleeding into the stomach and/or intestines may also have abnormal BUN levels. Numerous drugs also affect BUN by competing with it for elimination by the kidneys.

Normal Values: 7-20 mg/dl. Note that normal values may vary among different laboratories.

### What Abnormal Results Mean:

Greater-than-normal levels may indicate: Congestive heart failure; Excessive protein catabolism (possibly due to starvation); Excessive protein ingestion; Gastrointestinal bleeding; Hypovolemia (possibly due to burns or dehydration); Myocardial infarction (heart attack); Renal disease (for example, glomerulonephritis, pyelonephritis, and acute tubular necrosis); Renal failure; Shock; Urinary tract obstruction (for example, tumor, stones, and prostatic hypertrophy).

Lower-than-normal levels may indicate: Liver failure; Low protein diet; Malnutrition; Over-hydration.

Additional conditions under which the test may be performed: Acute nephritic syndrome; Alport syndrome; Atheroembolic renal disease; Chronic renal failure; Complicated UTI (pyelonephritis); Dementia due to metabolic causes; Diabetic nephropathy/sclerosis; Digitalis toxicity; End-stage renal disease; Epilepsy; Generalized tonic-clonic seizure; Goodpasture's syndrome; Hemolytic-uremic syndrome (HUS); Hepatorenal syndrome; IgM mesangial proliferative glomerulonephritis; Interstitial nephritis; Lupus nephritis; Malignant hypertension (arteriolar nephrosclerosis); Medullary cystic disease; Membranoproliferative GN I; Membranoproliferative GN II; Type 2 diabetes; Prerenal azotemia; Primary amyloid; Rapidly progressive (crescentic) glomerulonephritis; Secondary systemic amyloid; Wilms' tumor.

What the risks are: Excessive bleeding; Fainting or feeling light-headed; Hematoma (blood accumulating under the skin); Infection (a slight risk any time the skin is broken); Multiple punctures to locate veins.

For people with liver disease, the BUN level may be low even if the kidneys are normal. Some drugs affect BUN levels. Before having this test, the health care provider should be advised of which medications the patient is taking. Drugs that can increase BUN measurements include allopurinol, aminoglycosides, cephalosporins, chloral hydrate, cisplatin, furosemide, guanethidine, indomethacin, methotrexate, methyldopa, nephrotoxic drugs (for example, high-dose aspirin, amphotericin B, bacitracin, carbamazepine, colistin, gentamicin, methicillin, neomycin, penicillamine, polymyxin B, probenecid, vancomycin), propranolol, rifampin, spironolactone, tetracyclines, thiazide diuretics, and triamterene. Drugs that can decrease BUN measurements include chloramphenicol and streptomycin

The interaction between energy and protein within the body varies with the functional metabolic demand. The metabolic demand for energy is measured as the flow of carbon through the body, and the main determinant of variability within and between individuals is the level of physical activity. The metabolic demand for protein is measured as the flow of nitrogen through the body and the main determinant of variability within and between individuals is the rate of growth. Although the demands for nitrogen and carbon often move together in the same direction, this is not necessarily so. At marginal levels of energy intake, positive nitrogen balance may be defended in the face of a negative energy balance.

Nitrogen balance only represents a fraction of the intensity of the movement of nitrogen within the body, as there are two major internal cycles for nitrogen. The first, characterized as protein turnover, represents the movement of nitrogen as amino acids into and from proteins. The intensity and pattern of this movement vary with the pattern of the metabolic demand. The second, less clearly recognized, represents the movement of nitrogen from amino acids into urea, and the return of the urea-N to amino acid synthesis. The return of the urea-N requires the salvaging of nitrogen through the metabolic activity of the colonic microflora. Within the range of adequate protein intakes, the production of urea is unrelated to protein intake. The achievement of nitrogen balance appears to be dependent upon the salvage of urea-N, implying that the activity of the colonic microflora is an integral part of the mechanism through which nitrogen balance is maintained ordinarily. Nitrogen, amino acids and protein are not terms which can be used casually or interchangeably, and the movement of nitrogen through the body can only be measured directly with the use of nitrogen labels and not imputed indirectly from the use of carbon-labeled amino acids.

1.-11. (canceled)

12. A method for supporting organ functions and processes of the kidney and liver organs where a decline in quality or performance of said functions and processes have been brought about, by improving and treating various medical conditions of the kidney and liver organs, circulatory system, digestive system and cellular systems in human subeects or animal subjects, the method comprising:
metabolizing nitrogen and removing excess ammonia from said dioestive system, cellular system, kidney and liver organs of said human or animal subiects and balancing an acid pH level in said systems and organs of said human and animal subjects by administering a therapeutically effective amount of a nutritional dietary nutraceutical comprising a mastic gum herb, a processed extract of said mastic gum herb or a combination of said mastic gum herb and said processed extract of said mastic gum herb, said nutraceutical further comprising one or more minerals.

13. (canceled)

14. (canceled)

15. The method according to claim 12, wherein the one or more minerals include calcium and/or magnesium and/or a salt or ion thereof.

16. The method according to claim 15, wherein the calcium is obtained from coral.

17. (canceled)

18. The method according to claim 12, wherein when the subject being administered is a human, the method comprises:
administering the formed composition to said human, wherein the composition comprises up to about 20,000 milligrams of said mastic gum herb, said processed extract of said mastic gum herb or said combination of said mastic gum herb and said processed extract of said mastic gum herb and one or more active ingredients comprising up to about 20,000 milligrams of said one or more minerals.

19. The method according to claim 18, wherein the one or more minerals comprises up to about 20,000 milligrams of a magnesium and/or a salt thereof.

20. The method according to claim 18, wherein the one or more minerals comprises up to about 5000 milligrams of a vanadium and/or a salt thereof.

21. (canceled)

22. The method according to claim 12, wherein when the subject being administered is an animal, the method comprises administering up to about 20,000 milligrams of the mastic gum herb, said processed extract of said mastic gum herb or said combination of said mastic gum herb and said processed extract of said mastic gum herb and one or more active ingredients comprising up to about 20,000 milligrams of said one or more minerals.

23. The method according to claim 22, wherein the one or more minerals comprises up to about 20,000 milligrams of a magnesium and/or a salt thereof.

24. The method according to claim 22, wherein when the animal is being de-wormed, about 0.1% to about 50.0% of the nutraceutical further comprises one of or combinations of benzimidazole, pyrantel, and macrocyclic lactone based de-wormer compositions.
